(19) United States
(12) Patent Application Publication
(10) Pub. No.: US 2024/0122772 A1
(43) Pub. Date: Apr. 18, 2024

(54) METHOD OF MANAGING MENSTRUATION

(71) Applicant: Be-A Japan Inc., Tokyo (JP)

(72) Inventor: Kumi TAKAHASHI, Tokyo (JP)

(21) Appl. No.: 18/172,173

(22) Filed: Feb. 21, 2023

(30) Foreign Application Priority Data
Oct. 18, 2022 (JP) 2022-167246

(51) Int. Cl. A61F 13/42

(52) U.S. Cl. CPC A61F 13/42; A61F 2013/424; A61F 13/496

(57) ABSTRACT

To enable simpler and more efficient management of menstruation, this disclosure provides a method comprising the following steps: a processor acquires information pertaining to the amount of menstrual blood absorbed by an absorbent shorts s based on measurements taken by the measuring unit attached to the absorbent shorts; and the processor links the information pertaining to the amount of menstrual blood, and outputs it.

### TECHNICAL FIELD

The present disclosure relates specifically to methods of managing menstruation.

### BACKGROUND ART

In place of sanitary napkins, which were conventionally disposable, underwear-type absorbent panties that can be washed and used repeatedly are being developed as sanitary products. For example, Patent Literature 1 discloses technology related to easily washable sanitary absorbent panties.

### PRIOR ART LITERATURE

### Patent Literature

[Patent literature 1] JP6725958B1

### SUMMARY OF INVENTION

### Technical Problem

There is a need for simpler and more efficient management of menstruation.

Therefore, the present disclosure was made in view of the above problem, and its purpose is to provide a method that enables simpler and more efficient management of menstruation.

### Technical Solution

The present disclosure provides a method comprising the following steps: a processor acquires information pertaining to the amount of menstrual blood absorbed by an absorbent shorts s based on measurements taken by the measuring unit attached to the absorbent shorts; and the processor links the information pertaining to the amount of menstrual blood, and outputs it.

### Advantageous Effect

According to this disclosure, the management of menstruation can be performed in a simpler and more efficient manner.

### BRIEF DESCRIPTION OF DRAWINGS

FIG. 1 shows an example configuration of a menstruation management system 1 in accordance with an embodiment of the present disclosure.

FIG. 2 shows an example of the hardware configuration of the computer that realizes the server 10 of this embodiment.

FIG. 3 shows an example of the software configuration of the server 10.

FIG. 4 shows an example of the details of the water-absorbing shorts of the same embodiment.

FIG. 5 shows a table of an example of the wearing information DB 101 for this embodiment.

FIG. 6 shows a table of an example of the menstrual information DB 102.

FIG. 7 shows a flowchart diagram shows an example of a method of managing menstruation.

FIG. 8 shows an example of an information screen displayed on the user terminal 20.

FIG. 9 shows another example of the information screen displayed on the user terminal 20.

FIG. 10 shows the other example of the information screen displayed on the user terminal 20.

### DETAILED DESCRIPTION OF THE EMBODIMENTS

The following are detailed descriptions of suitable embodiments of the present disclosure with reference to the accompanying drawings. In this specification and the drawings, components having substantially identical functional configurations will be omitted from duplicated explanations by applying the same symbols.

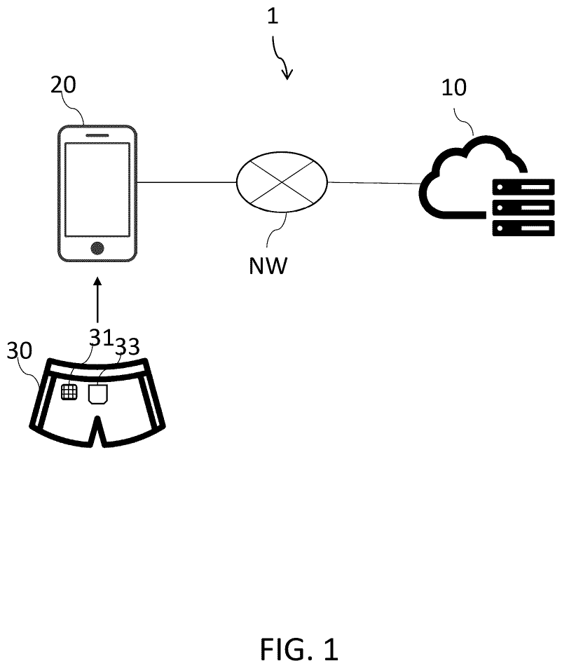

FIG. 1 shows an example configuration of a menstrual management system 1 in accordance with an embodiment of the present disclosure. As shown in the figure, the menstrual management system 1 of this embodiment has a cloud server 10 (hereinafter referred to as “server 10”), a user terminal 20, and an underwear 30. The server 10 and the user terminal 20 are communicatively connected via a network NW. This configuration is an example, and one configuration may combine or include other configurations. In this embodiment, the server 10 is configured to have the main functions described below, but some or all of such main functions may be provided by the user terminal 20. In other words, the menstrual management system 1 may comprise either server 10 or user terminal 20 alone, or server 10 and user terminal 20 may comprise the system jointly.

In this embodiment, the network NW is assumed to be the Internet. The network NW includes, for example, a public telephone network, a cellular telephone network, a wireless communication network, Ethernet (registered trademark), and so on.

Server 10 is, for example, a computer for managing the user's menstrual status, physical condition, menstruation-related schedule, etc., or for processing information output to the user based on such management information. Such server 10 may be managed, for example, by an organization that collects information on the user's menstrual information and provides services based on the collected data.

For example, server 10 may be a general-purpose computer such as a workstation or personal computer, or it may be logically realized by cloud computing.

The user terminal 20 is a terminal used, for example, by a user who manages her own menstrual information. Such a terminal can be operated by the user. For example, the user terminal 20 can be a smartphone, tablet computer, or personal computer, or the like.

The underwear 30 is, for example, women's sanitary absorbent panties. The construction and characteristics of such absorbent panties may be those disclosed, for example, in Patent No. JP6725958B2. The absorbent panties may be disposable or reusable.

Tag 31 is attached to the underwear 30. The tag 31 is an example of an identification unit. The tag 31 can be a tag based on RFID or NFC (Near Field Communication), for example. For example, The tag 31 can be provided to communicate with the user terminal 20. Specifically, by holding the user terminal 20 over the tag 31, the tag 31 is read and identified by the user terminal 20. The identification process here means the process related to the identification (such as shorts ID or the like) of the underwear 30 to which the tag 31 is attached by the user terminal 20. The result of the identification process of the tag 31 by the user terminal 20 can be transmitted to the server 10.

The example of the identification part is not limited to the tag 31 described above. For example, when a sensor and a device are incorporated in the underwear 30, and the sensor detects the user wearing the underwear 30, etc., the device can transmit the result of the identification process described above to user terminal 20 or server 10 via communication. The type of sensor is not particularly limited, and can be anything that can identify the wearing of the underwear 30 by the user, such as detecting contact or non-contact between the user and the underwear 30, or detecting bodily fluids, etc. emitted by the user. For example, such a device may be a device that is sewn into the underwear 30 and detects at least moisture with a sensor. In this case, the identification process may identify that the user has worn the underwear by detecting a predetermined amount of moisture by the sensor. The tag 31 may be a QR code (registered trademark) or other tag that enables identification processing by image recognition, but from the viewpoint of privacy protection, it is preferable to use a method that enables the tag 31 to be read by a method other than image recognition. However, identification processing by image recognition can be included in the scope of this technology.

The identification process may be a process to identify that the user has worn the underwear 30 based on the authentication result of the user information that identifies the user as “the user.” For example, the authentication of the user information may be performed at the time of the identification process of the underwear 30 to be worn for the first time, whereby the user is authenticated and the underwear 30 and the user information are linked, so that when the tag 31 of the underwear 30 is read during the identification process for the second and subsequent times, the tag 31 is read by the user terminal 20 linked with the user information. This can be achieved by querying the tag 31 and the user information so that identification processing is performed only when the tag 31 of the underwear 30 is read by the user terminal 20 to which the user information is linked. This allows only the user of the underwear 30 to access the user information, thereby improving security. When such underwear 30 is used by more than one person, the user information may be authenticated. This allows identification of who is using the underwear 30. This identification process may be omitted when it is not necessary to identify the user of the underwear in the measurement of the amount of menstrual blood described below.

The underwear 30 is also provided with a communication unit 33 (transmitter). The communication unit 33 transmits information pertaining to the amount of menstrual blood by the user of the underwear 30, such as resistance values or the like measured by the measurement unit described below, to the user terminal 20.

The following is an overview of the menstrual management system 1. A user, for example, wears underwear 30 with a tag 31 on a menstrual period day. At that time, the user brings the user terminal 20 close to the tag 31 to perform the identification process. Then, for example, a screen indicating that the user has worn the underwear 30 (or a screen confirming whether or not the user has worn the underwear 30) may be displayed on the user terminal 20. Also, for example, because such identification processing shows that the user has worn the underwear 30, the server 10 can record that the user is in a menstrual state based on the results of such identification processing. Furthermore, if the user turns on the communication unit 33 and establishes communication between the user terminal 20 and the communication unit 33 (e.g., Bluetooth (registered trademark)) on the setting screen of the user terminal 20, the measurement unit connected to the communication unit 33 will, at regular intervals (e.g., for the purpose of maintaining the charge level of the communication unit 33), perform a measurement process to measure the amount of menstrual bleeding in the underwear 30.

The communication unit 33 transmits information pertaining to the measured amount of menstrual blood to the user terminal 20, and via the user terminal 20, the server 10 obtains information pertaining to the amount of menstrual blood and transmits the information for output to the user terminal 20. The details of this system are described below.

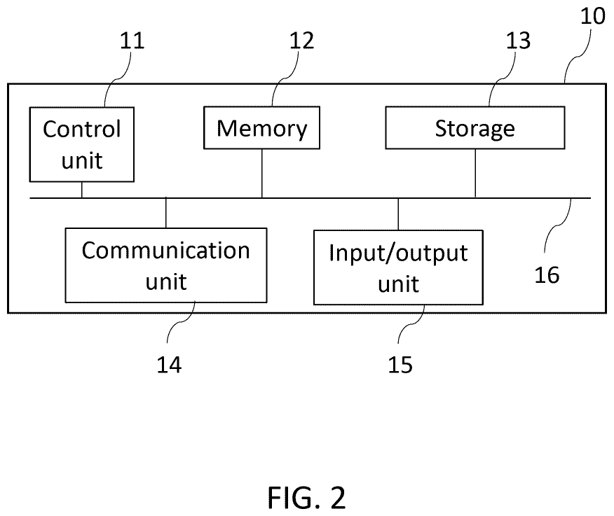

FIG. 2 shows an example of the hardware configuration of the computer that realizes the server 10. The computer is equipped with at least a control unit 11, memory 12, storage 13, communication unit 14, and input/output unit 15. These are electrically connected to each other through a bus 16.

The control unit 11 is an arithmetic unit that controls the operation of the entire server 10, controls the transmission and reception of data between each element, and performs information processing necessary for application execution and authentication/measurement processing, and the like. For example, the control unit 11 is a processor such as a CPU (Central Processing Unit), which executes programs, etc. stored in the storage 13 and deployed in the memory 12 to perform each information processing.

The memory 12 includes main memory comprising a volatile storage device such as DRAM (Dynamic Random Access Memory) and auxiliary memory comprising a non-volatile storage device such as flash memory or HDD (Hard Disc Drive). The memory 12 is used as a work area, etc. for control unit 11 and also stores BIOS (Basic Input/Output System), which is executed when server 10 is started, and various setting information, etc.

The storage 13 stores various programs such as application programs. A database storing data used for each process may be built in storage 13.

The communication unit 14 connects the server 10 to a network. The communication unit 14 communicates with external devices directly using, for example, wired LAN (Local Area Network), wireless LAN, Wi-Fi (Wireless Fidelity, registered trademark), infrared communication, Bluetooth (registered trademark), short-range or non-contact communication or through a network access point.

The input/output unit 15 is, for example, an information input device such as a keyboard, mouse, touch panel, etc., and an output device such as a display or the like.

The bus 16 is commonly connected to each of the above elements and transmits, for example, address signals, data signals, and various control signals.

The hardware configuration of the computer, smartphone, or other terminal that realizes the user terminal 20 in this embodiment is similar to the example of the hardware configuration of the server 10 shown in FIG. 2, so the description is omitted.

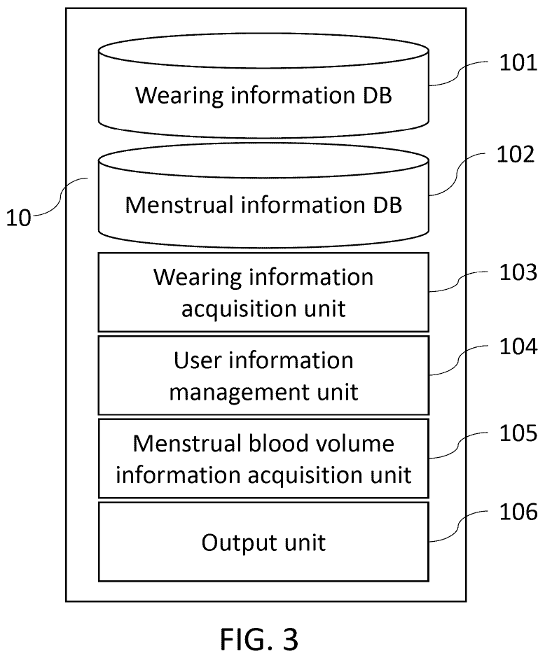

FIG. 3 shows an example of the software configuration of the server 10. The server 10 is equipped with a wearing information DB 101, a menstrual information DB 102, a wearing information acquisition unit 103, a user information management unit 104, a menstrual blood volume information acquisition unit 105, and an output unit 106. The wearing information acquisition unit 103, the user information management unit 104, the menstrual blood volume information acquisition unit 105, and the output unit 107 can be realized by the control unit 11 reading a program stored in the storage 13 into the memory 12 and executing it. The wearing information DB 101 and the menstrual information DB 102 can be realized as part of the storage space provided by the memory 12 and the storage 13. The wearing information DB 101 and the organizing information DB 102 can be realized as part of an external storage area provided by one or more servers other than the server 10.

The wearing information DB 101 is a database that stores wearing information obtained by the wearing information acquisition unit 103 based on the tag 31 identification process.

The menstrual information DB 102 is a database that stores menstrual blood volume information obtained by the menstrual blood volume information acquisition unit 105 based on the measurement process of the measurement unit attached to the underwear 30, which will be described later.

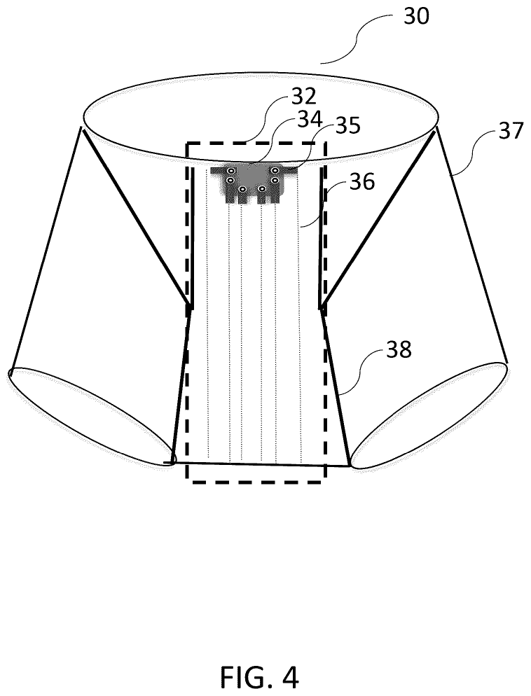

FIG. 4 shows an example of a detail of the water-absorbing panties. As shown in FIG. 4, the underwear 30 has an absorbent body 38 capable of absorbing and retaining menstrual blood and urine, which is provided in a body part 37 of the absorbent panty. Here, an absorbent body 2 is attached to the inside (skin side) of the body part 37. The absorbent 2 has a water-absorbing material and can absorb liquid discharged from the body, such as menstrual blood or urine, and prevent it from seeping into the main body 37. The absorbent 38 can be made of a multi-layered structure of multiple materials. For example, knit or non-woven fabrics such as cotton, cotton pile, antibacterial deodorant acrylic, water-absorbing acrylic, water-absorbing polyester, cupra, rayon, etc. can be used as materials having absorption functions, and a mixed material consisting of rayon and acrylic can be used. The water absorbing capacity of absorbent 38 can be adjusted according to the degree of the amount of menstrual blood or urine, for example, it should be able to absorb 10 ml to 300 ml of liquid. The underwear 30 also has a measuring unit 32 attached to the absorbent body 38 to measure the amount of menstrual blood of the user of the underwear 30. The measuring unit 32 has a base 34 for attaching the communication unit 33, a ribbon electrode 35 connected to the base 34 via a hook or the like, and a plurality of thread-like fibers 36 with, for example, a nylon core material and a conductive metal such as silver coating around it, which are connected to the ribbon electrode 35 by sewing or other means. The measuring unit 32 can measure the amount of menstrual blood absorbed by the absorbent 38 based on the resistance of the electricity flowing through the electrodes 35 and fibers 36, which are in contact with the absorbent 38. Thus, by using skin-friendly materials such as fibers and ribbon electrodes as the measuring unit, the user of the underwear 30 can measure the amount of menstrual blood just by wearing the underwear as usual, and since the communication unit 33 and base 34 are located on the upper front side of the underwear 30, the user can wear the underwear 30 without any discomfort. In addition, by making the communication unit (transmitter) removable from the underwear 30, the user can usually remove the transmitter and wash the underwear 30.

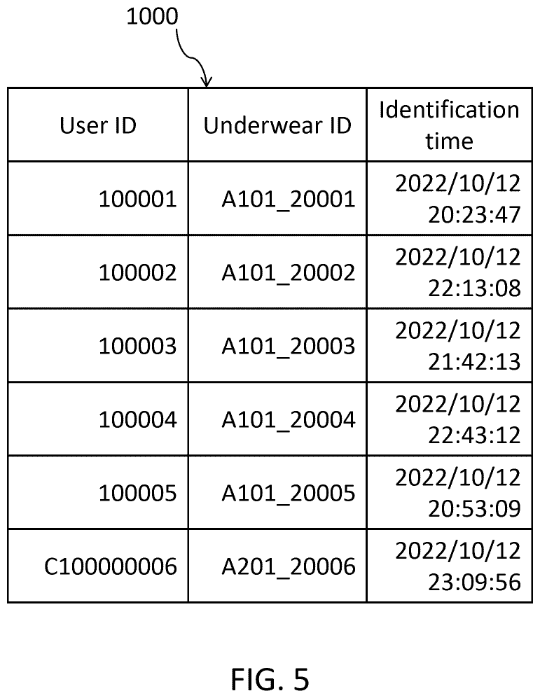

FIG. 5 is a table showing an example of the wearing information DB 101. The table 1000 of wearing information shown in FIG. 5 may include information on the user ID of the user who wears the underwear 30, the shorts ID which is the product ID of the underwear 30, and the identification time. The wearing information may include information other than the example shown in FIG. 4.

The user ID is an identifier that uniquely identifies a user who uses the underwear 30 and uses an application installed on the user terminal 20. The shorts ID is an identifier that uniquely identifies the underwear 30 to which the tag 31 is attached, which can be obtained by reading the tag 31 with the user terminal 20. The identification time is the time at which the identification process was performed.

Such table 1000 can be managed by tying each user to shorts ID. This allows the underwear used by a user to be identified, and the identification time can be used to manage that the user has worn the underwear.

The menstrual information DB 102 is a database that manages information about the user's menstruation (menstrual information). For example, the menstrual information DB 102 stores the amount of menstrual blood in the user's period, schedule information that is information about the schedule of menstruation, and other information as menstrual information.

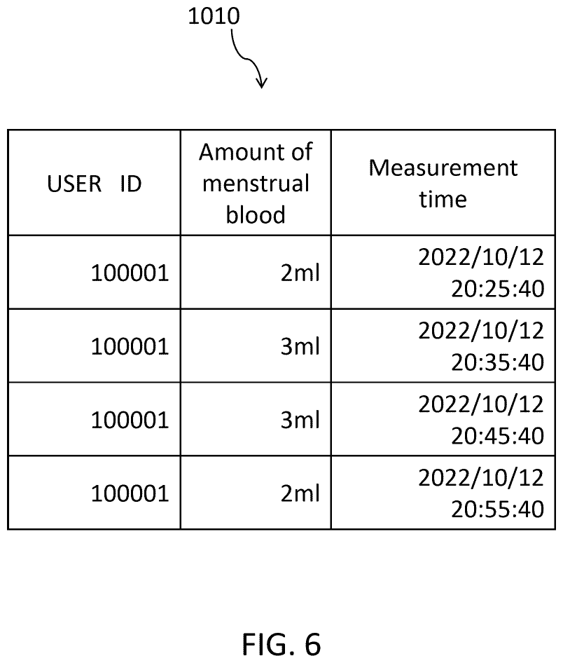

FIG. 6 is a table showing an example of the menstrual information DB 102. The table 1010 of schedule information shown in FIG. 6 can include information on the user ID, the amount of menstrual blood, and the time of measurement. The amount of menstrual blood may be managed as a resistance value, as described above, although it is indeed a numerical value calculated based on a predetermined formula for the resistance value of electricity flowing through electrodes. The menstrual information may include information other than the example shown in FIG. 6.

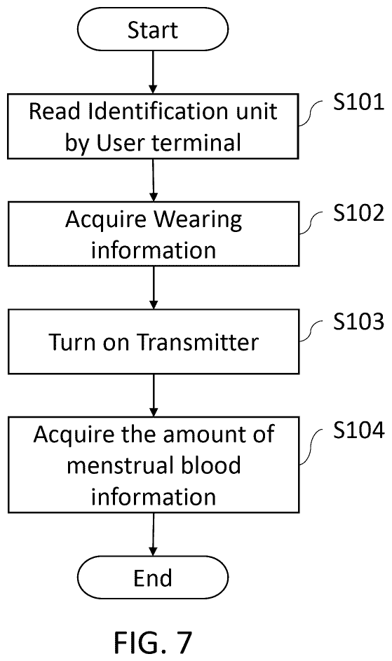

FIG. 7 is a flowchart diagram of an example of a method of managing menstruation.

First, the user reads the tag 31 (identification unit) attached to the underwear 30 using the user terminal 20 when wearing the underwear 30 (step S101). When the tag 31 is read, a confirmation screen is displayed on the user terminal 20 asking whether or not to perform the identification process (i.e., whether or not to wear the underwear 30). Such processing can be performed by an application (including one via a web server) running on the user terminal 20 when the tag 31 is read.

Next, the wearing information acquiring unit 102 acquires information on the wearing of the underwear 30 through the identification process, and the user information management unit 104 records the information pertaining to the shorts ID that identifies the underwear 30 and the identification time when the identification process was performed as wearing information, in connection with the user ID that identifies the user who wears the underwear 30 and uses the application (step S102). Such check-in date may be recorded as the day of the menstruation.

Next, the user attaches the communication unit 33 (transmitter) to the base 34 on the underwear 30, turns on the transmitter 33 (e.g., press and hold the power button), and configures the setting screen of the application installed on the user terminal 20 to establish communication (e.g., Bluetooth) between the user terminal 20 and the transmitter. The transmitter 33 then sends information to start measuring the amount of menstrual blood via the user terminal 20, and the menstrual blood volume information acquisition unit 102 acquires information to that effect (step S103). Here, the power of the transmitter 33 is turned on, and an electric current flows through the conductive fibers 36 via the electrodes 35 in contact with the transmitter 33. When the measuring unit 32 measures the resistance value of the electricity flowing through the fiber 36 sewn into the absorbent body 38 attached to the body 37 of the underwear 30, the menstrual blood volume information acquisition unit 105 acquires information pertaining to the measured resistance value, or information pertaining to the menstrual blood volume calculated based on a formula representing the correlation between the resistance value and the menstrual blood volume value, along with the measurement time, via user terminal 20 (step S104). Considering the amount of charge in the transmitter 33, for example, the information pertaining to the amount of menstrual blood measured continuously for a predetermined period of time (100 seconds) may be acquired after a certain period of time (e.g., 1 minute). The user information management unit 104 records the acquired information pertaining to the amount of menstrual blood in the menstrual information DB, linking it to the user ID and measurement time.

The output unit 106 generates the information related to the amount of menstrual blood obtained in this way as screen information that can be displayed on the user terminal 20, and transmits it to the user terminal 20. By measuring the amount of menstrual blood and presenting it to the user, the user can know the amount of menstrual blood and prepare for anemia or other physical problems. The output unit 106 can also generate a learning model by machine learning of the correlation between the amount of menstrual blood and gynecological diseases such as premenstrual syndrome, infertility, and menopausal disorders, and the correlation between the amount of menstrual blood stored in the server 10 and each disease, and can estimate the risk of each disease based on the information pertaining to the input menstrual blood volume.

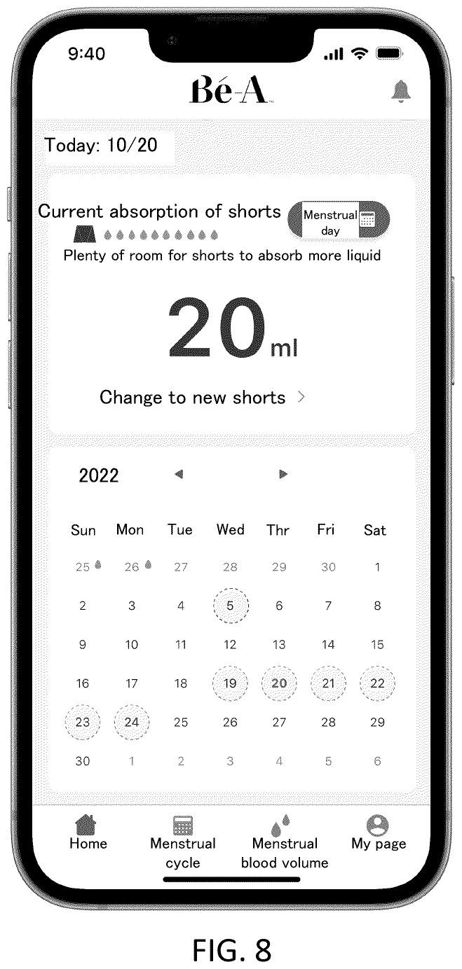

FIGS. 8 through 10 show examples of information pertaining to the amount of menstrual blood displayed on the user terminal 20.

As shown in FIG. 8, the total amount of menstrual blood measured on a particular date can be displayed. In addition to the measured amount of menstrual blood, information pertaining to the amount of menstrual blood that can be absorbed by the shorts identified in the identification process can also be displayed. When the ratio of the measured amount of menstrual blood to the amount of water absorbable menstrual blood (e.g., 300 ml in the previous example) increases, the output unit 106 can send a notification recommending that the user change to new shorts. As shown in FIG. 8, the calendar display can also show information related to the amount of menstrual blood corresponding to the date specified by the user. Among those dates, days with a large amount of menstrual blood (e.g., days with more than a predetermined amount) can be displayed in a form that allows them to be distinguished from other days (e.g., by highlighting the date or displaying it in a different color).

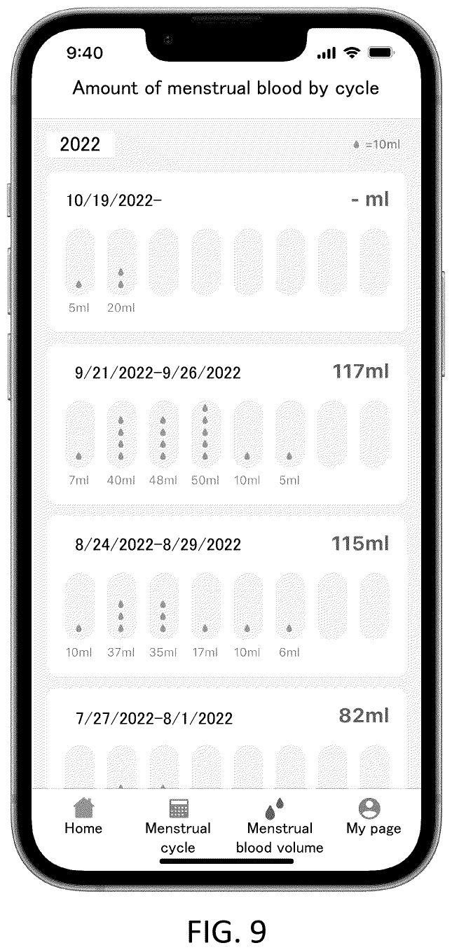

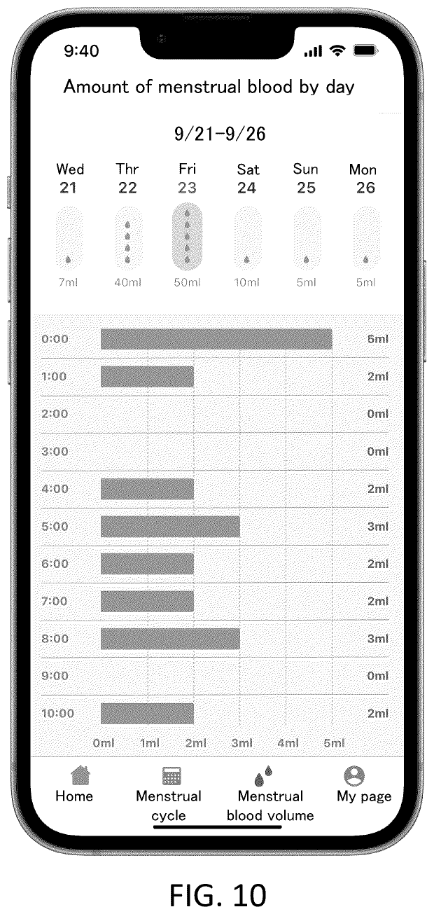

As shown in FIG. 9, the total amount of menstrual blood for each menstrual cycle can be displayed, and the amount of menstrual blood by date for each cycle can be replaced by symbols or figures so that the user can understand at a glance. Furthermore, as shown in FIG. 10, the amount of menstrual blood can be displayed by time of day for each date.

As explained above, according to the menstrual management system 1 of this embodiment, the timing of the user's menstruation can be automatically recorded by reading the tag 31 attached to the menstrual underwear 30. This allows the user to efficiently and easily manage her menstruation while using the menstrual underwear. According to the menstrual management system 1, not only can the measurement of the amount of menstrual blood during the user's menstruation, which has been difficult in the past, be realized, but the user can also measure the amount of menstrual blood without any burden by simply wearing underwear, which further makes the management of menstruation more efficient and convenient.

Although the suitable embodiments of the present disclosure have been described in detail with reference to the accompanying drawings, the technical scope of the present disclosure is not limited to such examples. It is clear that a person having ordinary knowledge in the technical field of the present disclosure can conceive of various examples of changes or modifications within the scope of the technical ideas described in the claims, which are naturally understood to be within the technical scope of the present disclosure.

The device described herein may be realized as a stand-alone device or by multiple devices (e.g., cloud servers) connected in part or in whole by a network. For example, the control unit 11 and the storage 13 of the server 10 may be realized by different servers connected by a network to each other.

The series of processes by the device described herein may be realized using software, hardware, and combinations of software and hardware. It is possible to create a computer program to realize each function of the server 10 in this embodiment and implement it on a PC or other device. A computer-readable recording medium in which such a computer program is stored can also be provided. The recording medium is, for example, a magnetic disk, optical disk, magneto-optical disk, flash memory, etc. The computer program described above may also be delivered over a network, for example, without using a recording medium.

The processes described herein using a flowchart diagram do not necessarily have to be performed in the order illustrated. Some processing steps may be executed in parallel. Additional processing steps may be employed, and some processing steps may be omitted.

The effects described herein are only descriptive or exemplary and not limiting. In other words, the technology of the present disclosure may produce other effects that are obvious to those skilled in the art from the description herein, either together with or in place of the above effects.

The following configurations are also within the technical scope of this disclosure.

[Item 1]

A method comprising the following steps: a processor acquires information pertaining to the amount of menstrual blood absorbed by an absorbent shorts s based on measurements taken by the measuring unit attached to the absorbent shorts; and
the processor links the information pertaining to the amount of menstrual blood, and outputs it.

[Item 2]

The method according to item 1, wherein the measuring unit has an electrode unit attached to contact a water absorbing body provided in the body of the absorbing shorts.

[Item 3]

The method according to item 2, having a communication unit that is connected to the electrode unit and transmits information pertaining to the amount of menstrual blood absorbed by the absorbent shorts.

[Item 4]

The method according to item 2, wherein a resistance value of the electrode unit is substituted for the amount of menstrual blood absorbed by the absorbing body to generate information pertaining to the amount of menstrual blood.

[Item 5]

The method according to item 1, wherein the information pertaining to the amount of menstrual blood is obtained for each predetermined period of time, and the total value of the information pertaining to the amount of menstrual blood obtained for each predetermined period of time is output.

[Item 6]

The method according to item 1, wherein information pertaining to the wearing of the absorbent shorts is obtained by an identification process based on an identification part attached to the absorbent shorts.

### DESCRIPTION OF REFERENCE NUMERALS

1 Menstrual management system
10 Server
20 User terminal
30 Underwear (absorbent shorts)
31 Tag (identification unit)
101 Wearing information DB
102 Menstruation information DB
103 Wearing information acquisition Unit
104 User information management unit
105 Blood volume information acquisition unit
106 Output unit

1. A method comprising the following steps: a processor acquires information pertaining to the amount of menstrual blood absorbed by an absorbent shorts s based on measurements taken by the measuring unit attached to the absorbent shorts; and
the processor links the information pertaining to the amount of menstrual blood, and outputs it.

2. The method according to claim 1, wherein the measuring unit has an electrode unit attached to contact a water absorbing body provided in the body of the absorbing shorts.

3. The method according to claim 2, having a communication unit that is connected to the electrode unit and transmits information pertaining to the amount of menstrual blood absorbed by the absorbent shorts.

4. The method according to claim 2, wherein a resistance value of the electrode unit is substituted for the amount of menstrual blood absorbed by the absorbing body to generate information pertaining to the amount of menstrual blood.

5. The method according to claim 1, wherein the information pertaining to the amount of menstrual blood is obtained for each predetermined period of time, and the total value of the information pertaining to the amount of menstrual blood obtained for each predetermined period of time is output.

6. The method according to claim 1, wherein information pertaining to the wearing of the absorbent shorts is obtained by an identification process based on an identification part attached to the absorbent shorts.
